(19) United States
(12) Patent Application Publication
(10) Pub. No.: US 2025/0342957 A1
(43) Pub. Date: Nov. 6, 2025

(54) LOCATION BASED TOILETING

(71) Applicant: Raja Singh Tuli, Montreal (CA)

(72) Inventor: Raja Singh Tuli, Montreal (CA)

(21) Appl. No.: 19/196,111

(22) Filed: May 1, 2025

Related U.S. Application Data
(60) Provisional application No. 63/641,698, filed on May 2, 2024.

(51) Int. Cl. G16H 40/67; G01S 5/02

(52) U.S. Cl. CPC G16H 40/67; G01S 5/02521; G01S 2205/10

(57) ABSTRACT

Inside a building equipped with a number of WIFI routers, an object carrying a portable unit is located based on WIFI signal profile that is one or more of WIFI signal strengths from the WIFI routers. The WIFI signal profile of the portable unit carried on the object is determined and compared to the WIFI signal profiles for specific locations in the building, and the object is determined to be located at or close to the one specific location whose WIFI signal profile matches best the WIFI signal profile of the portable unit.

### CROSS-REFERENCE TO RELATED APPLICATIONS

This application claims priority to U.S. provisional patent application No. 63/641,698, entitled “LOCATION BASED TOILETING,” filed on May 2, 2024. The content of this U.S. provisional patent application is hereby incorporated by reference in its entirety for all purposes.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR OR A JOINT INVENTOR

Not Applicable

### BACKGROUND OF THE INVENTION

### Field of the Invention

The present disclosure relates to locating an object such as a person e.g. a resident or patient inside a building e.g. in a care facility, and in particular to locating with WIFI signature.

The present disclosure further relates to system and method for recording/registering action e.g. based on the action-related location. In the context of the present disclosure, an action may be identified as a sequence of motions.

The present disclosure also relates to automatically recording or registering an action on or by an object e.g. a resident or patient or a caregiver inside a building e.g. in a care facility, and in particular to automatically recording or registering based on WIFI signal profile.

### Description of Related Art

In care facilities such as nursing centers, the majority of residents and/or patients suffer from incontinence and/or dementia. With respect to incontinence, an absorbent article such as diaper may be used in order to allow its wearer to urinate or defecate without the use of a toilet, and a portable unit e.g. alerting unit such as a pod may be attached (e.g. releasably attached) to (such as cliped on) the absorbent article to detect and measure the wetness in the absorbent article and provide alert accordingly.

With dementia like Alzheimer, a resident and/or patient suffers from the impaired ability to remember, think, or make decisions that interferes with doing everyday activities. For example, a person with dementia may suffer from memory loss, e.g. she/he may forget what they just did even only 2 minutes ago. As another example, she/he often does not know what to do next. In a care facility, the resident/patient with dementia sometimes gets out of bed, starts walking, and then gets lost in the care facility.

In general, a care facility is located in a large building with a number of rooms and hallways. A room in a care facility is assigned to and occupied by one resident or assigned to and shared by more than one residents. In such a care facility, a lost resident needs to be located as soon as possible, in order to avoid any potential accident and injury. However, inside a building, conventional locating techniques such as GPS does not work very well. Therefore, there is a need for a locating technique to timely and accurately locate a person e.g. a resident or patient inside a building e.g. in a care facility.

A care facility e.g. nursing center is often subsidized by the government or some program or organization, e.g. receives revenue from the government or some program or organization, but under certain condition. In general, the government or program or organization provides subsidies to a care facility for some specific services or assistances provided to the residents or patients, e.g. assistance for toileting, for feeding, and etc. Therefore, the care facility, in particular its employees, needs to keep record after each service or assistance or action as required by the subsidies. However, it is possible and often happens that the care facility, in particular its employee, forgets to keep record those actions/services/assistances, which might result in some problem or difficulty with subsidies. Therefore, there is a need for automatically recording or registering an action on or by an object e.g. a resident or patient or a caregiver inside a building e.g. in a care facility.

### BRIEF SUMMARY OF THE INVENTION.

Embodiments are presented herein of, inter alia, a locating method to locate a person e.g. a resident or patient inside a building e.g. a care facility.

In an aspect of the present disclosure, a method is provided for locating a portable unit based on WIFI signature. The method comprises: the portable unit determining and sending its WIFI signature to a server, and the portable unit's location being determined based on the WIFI signature. In an embodiment of the present disclosure, when detecting a motion, the portable unit determines and sends its WIFI signature more frequently than when detecting no motion.

In another aspect of the present disclosure, a method is provided for locating a portable unit based on WIFI signature. The method comprises: the portable unit starting to determine and send its WIFI signature to a server when detecting an off-bed condition, and the portable unit's location being determined based on the WIFI signature.

In a further aspect of the present disclosure, a method is provided for locating a portable unit based on WIFI signature. The method comprises: the portable unit determining and sending its WIFI signature to a server, and the portable unit's location being determined based on the WIFI signature. In an embodiment of the present disclosure, the frequency of the portable unit determining and sending its WIFI signature depends on the server.

In an additional aspect of the present disclosure, a method is provided for locating a portable unit based on WIFI signature. The method comprises: the portable unit determining and sending its WIFI signature to a server, and the server determining the portable unit's location based on the WIFI signature received from the portable unit.

In another aspect of the present disclosure, a method is provided for automatically recording an action on or by an object based on WIFI signature. The object carries a portable unit, and the method comprises: monitoring the WIFI signature of the portable unit; determining the trajectory of the object based on the WIFI signature as monitored; comparing the determined trajectory of the object to a predetermined trajectory of the action; and recording the action if the determined trajectory substantially matches the predetermined trajectory. In the context of the present disclosure, the term trajectory (also referred to therein as motion path) refers to a set of movements or a set of actions. In an embodiment of this aspect, the purpose of the recording is to report the recorded action in order for funding.

In a further aspect of the present disclosure, a system is provided for automatically recording an action on or by an object based on WIFI signature. The system comprises: a portable unit carried on the object; and a recording device. The recording device is configured to determine the trajectory of the object based on the WIFI signature of the portable unit, compare the determined trajectory of the object to a predetermined trajectory of the action, and record the action if the determined trajectory substantially matches the predetermined trajectory.

In another aspect of the present disclosure, a method is provided for automatically recording an action on or by an object. A portable unit is carried on the object, and the method comprises: detecting the action with aid of the portable unit; and recording the action (e.g. in database). The action may comprise change of absorbent article, or cleaning the object, or the object cleaning another object. In an embodiment, an action may be identified as a sequence of motions.

In an additional aspect of the present disclosure, a system is provided for automatically recording an action on or by an object. The system comprises: a portable unit carried on the object, configured to determine the action; and a recording device configured to record the action (e.g. in database). The action may comprise change of absorbent article, or cleaning the object, or the object cleaning another object. In an embodiment, an action may be identified as a sequence of motions.

In another aspect of the present disclosure, a portable unit attachable to an absorbent article is provided. The portable unit comprises: a wetness detection circuit configured to detect wetness in the absorbent article periodically while the portable unit is attached to the absorbent article, and a WIFI circuit configured to determine and send WIFI signature. The WIFI circuit is woken up from Sleep mode under a first condition, and returns to Sleep mode under a second condition.

In an embodiment of this aspect, the first condition comprises one of: the absorbent article with the portable article being attached thereto is being put on or taken off, the portable unit is being detached from the absorbent article, an alert needs to be sent via WIFI based on the wetness detected by the wetness detection circuit, or a motion is determined. In an embodiment of this aspect, the second condition comprises one of: a different motion is determined, or no motion is determined at least for a certain period of time.

In another aspect of the present disclosure, a portable unit attachable to an absorbent article is provided. The portable unit comprises a WIFI circuit configured to determine and send WIFI signature. The WIFI circuit is woken up from Sleep mode when the portable unit is detached from the absorbent article, then determines and sends WIFI signature periodically with a period increasing over time. And the WIFI circuit returns to Sleep mode after a certain period of time following its wake up.

In a further aspect of the present disclosure, a method is provided for locating an object inside a building based on WIFI signature. The building is equipped with a number of WIFI routers, and the WIFI signature is one or more of WIFI characteristics with respect to one or more of the WIFI routers. The method comprises: determining the WIFI signature of a portable unit carried on the object; comparing the WIFI signature of the portable unit to each of the WIFI signatures of specific locations in the building; and determining the object to be located at or close to the one specific location whose WIFI signature matches best the WIFI signature of the portable unit.

In an embodiment of this aspect, the WIFI signature of the portable unit is determined periodically after specific motion(s) of the portable unit or of the object is detected. In an embodiment of this aspect, the period for determining the WIFI signature of the portable unit increases over time. And in an embodiment of this aspect, the building comprises a care facility, and the object comprises a resident in the care facility.

In another aspect of the present disclosure, a system is provided for locating an object inside a building based on WIFI signature. The system comprises: a number of WIFI routers deployed in the building, wherein the WIFI signature is one or more of WIFI characteristics with respect to one or more of the WIFI routers; a portable unit carried on the object; and a locating device configured to obtain the WIFI signature of the portable unit, compare the WIFI signature of the portable unit to each of the WIFI signatures of specific locations in the building, and determine the object to be located at or close to the one specific location whose WIFI signature matches best the WIFI signature of the portable unit.

In an embodiment of this aspect, the locating device obtains the WIFI signature of the portable unit periodically after specific motion(s) of the portable unit or of the object is detected. In an embodiment of this aspect, the period for obtaining the WIFI signature of the portable unit increases over time. And in an embodiment of this aspect, the building comprises a care facility, and wherein the object comprises a resident in the care facility.

In a further aspect of the present disclosure, a method is provided for automatically recording an action on or by an object inside a building based on WIFI signature. The building is equipped with a number of WIFI routers, the WIFI signature is one or more of WIFI characteristics with respect to one or more of the WIFI routers, and the object carries a portable unit. The method comprises: initiating a monitoring on the WIFI signature of the portable unit in response to specific motion(s) of the object or in response to a signal from the object; determining the trajectory of the object based on the WIFI signature as monitored; comparing the determined trajectory of the object to a predetermined trajectory; and recording the action if the determined trajectory substantially matches the predetermined trajectory.

In an embodiment of this aspect, the monitoring on the WIFI signature of the portable unit is terminated after the action is recorded and/or when the object is back to its previous location when the monitoring was initiated. In an embodiment of this aspect, the trajectory of the object is determined by comparing the WIFI signature as monitored to the WIFI signature(s) of specific location(s) in the building. In an embodiment of this aspect, the building comprises a care facility, and wherein the object comprises a resident in the care facility. And in an embodiment of this aspect, the action on the object comprises assistance for the object with toileting, or walking/moving the object.

In a further aspect of the present disclosure, a system is provided for automatically recording an action on or by an object inside a building based on WIFI signature. The system comprises: a number of WIFI routers deployed in the building, wherein the WIFI signature is one or more of WIFI characteristics with respect to one or more of the WIFI routers; a portable unit carried on the object; and a recording device configured to obtain the WIFI signature of the portable unit as monitored in response to specific motion(s) of or a signal from the object, determine the trajectory of the object based on the WIFI signature as monitored, compare the determined trajectory of the object to a predetermined trajectory, and record the action if the determined trajectory substantially matches the predetermined trajectory.

In an embodiment of this aspect, the monitoring on the WIFI signature of the portable unit is terminated after the action is recorded and/or when the object is back to its previous location when the monitoring was initiated. In an embodiment of this aspect, the record device determines the trajectory of the object by comparing the WIFI signature as monitored to the WIFI signature(s) of specific location(s) in the building. In an embodiment of this aspect, the building comprises a care facility, and wherein the object comprises a resident in the care facility. And in an embodiment of this aspect, the action on the object comprises assistance for the object with toileting, or walking/moving the object.

This summary is intended to provide a brief overview of some of the subject matter described in this document. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The various preferred embodiments of the present invention described herein can be better understood by those skilled in the art when the following detailed description is read with reference to the accompanying drawings. The components in the figures are not necessarily drawn to scale and any reference numeral identifying an element in one drawing will represent the same element throughout the drawings. The figures of the drawing are briefly described as follows.

FIG. 1 schematically illustrates a part of an exemplary floor plan of a care facility in which a method, device, and system according to the present disclosure may be employed.

While the features described herein are susceptible to various modifications and alternative forms, specific embodiments thereof are shown by way of example in the drawings and are herein described in detail. It should be understood, however, that the drawings and detailed description thereto are not intended to be limiting to the particular form disclosed, but on the contrary, the intention is to cover all modifications, equivalents and alternatives falling within the spirit and scope of the subject matter as defined by the appended claims.

### DETAILED DESCRIPTION OF THE INVENTION

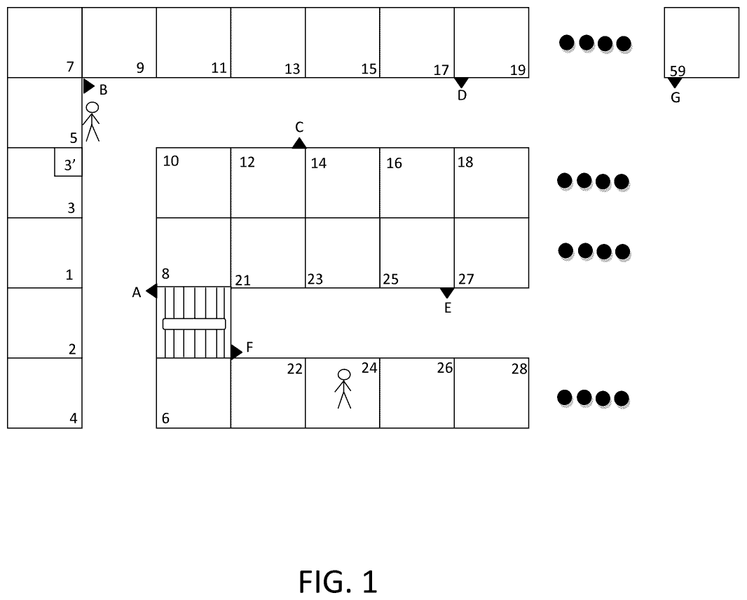

In general, there are plenty of rooms in a care facility that are assigned to the residents or patients. A room in a care facility may be assigned to and occupied by one resident or be assigned to and shared by more than one residents. FIG. 1 schematically illustrates a part of an exemplary floor plan of a care facility, according to an embodiment of the present disclosure. In FIG. 1, rooms 1, 2, 3, . . . , 59, and hallway as well as staircase are schematically illustrated in a care facility, as a non-limiting example. In addition, in order to provide a local area network, several WIFI access points e.g. routers are provided at different locations in a care facility. As an non-limiting example, seven WIFI routers A, B, C, D, E, F, and G are illustrated as triangles in FIG. 1.

In a care facility, each location e.g. each room, each bed, and each toilet has its respective unique WIFI profile, such as WIFI signature/characteristic (e.g. with respect to at least one of the WIFI access points), e.g. signal strength (i.e. Received Signal Strength Indicator (RSSI)) from at least one of the WIFI routers, Time-of-Flight (ToF) with respect to at least one of the WIFI routers, Angle of Arrival (AoA) with respect to at least one of the WIFI routers. In the following description, WIFI signal strength (e.g. RSSI) is used as WIFI profile/signature/characteristic, however those skilled in the art will appreciate that this is only for illustrative purpose, and any now known or later developed WIFI-based locating method as well as the associated WIFI features or characteristics may be used with the present disclosure, without departing from the spirit and scope of the present disclosure.

For example, room No. 2 has a strongest signal strength from WIFI router A, a slightly weaker signal strength from WIFI router B, a more weaker signal strength from WIFI router F, . . . a substantially weaker signal strength from WIFI router D, and a extremely weak or even no signal strength from WIFI router G. It is also understood that different rooms will have different signal strengths from a WIFI router, because of their different relative locations with that WIFI router. As an example, with respect to a WIFI router C, room No. 12 has a strongest signal strength, room No. 14 has a second strongest signal strength, room No. 13 has a third strongest signal strength, . . . , and so on. So, for a specific room, the complete set of all the signal strengths from all the WIFI routers in a care facility, or at least part of the set (e.g. one or more of the signal strengths from WIFI routers) may constitute its unique WIFI signature (also referred to therein as WIFI profile), which may be used to identify that specific room and/or its location. Similarly, each bed and each toilet in a care facility have their unique WIFI signatures (or WIFI profiles).

In a care facility such as nursing centers, the majority of residents and/or patients may suffer from incontinence, and therefore the residents and/or patients are required to wear an absorbent article with a portable alerting unit attached thereon. A portable alerting unit is configured to be e.g. releasably attached to an absorbent article and to detect and measure the wetness in the absorbent article and provide alert accordingly. In order to identify its wearer and/or her/his room and/or bed, a portable alerting unit is assigned a unique identifier, which enables the facility e.g. its personnel such as caregiver and/or its system (e.g. a server) to know which room and/or bed and whom shall be visited to change the absorbent article when a portable alerting unit alerts. That is, with the unique identifier assigned to a portable alerting unit, the resident or patient who wear that portable alerting unit, as well as her/his room and/or her/his bed if there are more than one bed in her/his room, can be identified.

As an example, a database may be provided for a facility, which registers or stores the information/data on facility, e.g. on residents/patients, portable alerting units, rooms, beds, toilets in the facility, such as e.g. the WIFI signature/profile of each room and/or each bed and/or each toilet in the facility, the association between a resident/patient and her/his portable alerting unit, and the association between a resident/patient and/or her/his portable alerting unit on one hand and her/his room and/or bed and/or toilet on the other hand, and etc. These information/data may be input into the database by a facility personnel such as nurse or caregive at a certain point in time, e.g. in the beginning when the system is set up or initialized.

It is appreciated that the particular location of a room and a bed and a toilet in the facility may not be required to be explicity registered/stored in the database. On the contray, the database may register/store instead an identifier of a room (e.g. a room number) and/or an identifier of a bed (e.g. a bed number) and/or an identifier of a toilet (e.g. a toilet number), as an example. That is, from the database, it is not necessarily known where a room (e.g. with a room number) and a bed (e.g. with a bed number) and a toilet (e.g. with a toiletnumber) is located physically in the facility. Instead, it can only obtain from the database which resident/patient (e.g. denoted by an identifier, e.g. resident identier) is assigned which room (e.g. a room number) and/or which bed (e.g. a bed number) and/or which toilet (e.g. a toilet number).

As for the particular physical location of a bed and/or a room and/or a toilet in the facility, it can be identified or determined based on its occupant (e.g. a corresponding resident/patient)'s daily behaves in the facility, in an embodiment of the present disclosure. As an example, the particular location of a room (as denoted in the database by a room number) assigned to a resident/patient (as denoted in the database by a resident number) may be identified or determined e.g. based on the fact that the resident/patient (i.e. its occupant) and her/his portable alerting unit (as denoted in the databased by a unit number) stay in her/his room for all night time and/or for a substantial period (e.g. most or nearly all) of her/his day time and based on the WIFI signature/profile of her/his portable alerting unit at those periods of time (i.e. when in her/his room). Similarly, e.g. based on the fact that a resident/patient (i.e. an occupant, e.g. as denoted in the database by a resident number) stays in her/his bed (as denoted in the database by a bed number) at night for a long period of time without much motion and based on the WIFI signature/profile of her/his portable alerting unit (as denoted in the database by a unit number) when in her/his bed (i.e. at night without much movement for a long period), the particular location of the bed can be identified or determined, as an non-limiting example. Also, the particular location of a toilet (as denoted in the database by a toilet number) associated with a resident/patient (as denoted in the database by a resident number) may be identified or determined e.g. based on the fact that the toilet is usually located in her/his room and that the resident/patient (i.e. its occupant) goes to her/his toilet several times (e.g. once, twice, or three times) a day, stays every time in her/his toilet for a certain period of time (e.g. few minutes, or up to half an hour), and then comes back to her/his bed, and based on the WIFI signature/profile of her/his portable alerting unit when in her/his toilet, as an non-limiting example. In a similarly manner, the WIFI signature/profile of each room and/or each bed and/or each toilet in the facility may be determined based on its respective occupant (e.g. resident/patient)'s daily behaves (routine) in the facility with aid of her/his respective portable alerting. It is to be noted that, the particular location and WIFI signature/profile of rooms, beds, and toilets may be input into and/or calibrated in the database by a facility personnel, in an embodiment of the present disclosure.

Similarly to the rooms and beds in a facility, each portable alerting unit in a care facility has its WIFI signature (or WIFI profile) as well. However, since a portable alerting unit moves with its wearer, its WIFI signature (or WIFI profile) will change with the movement of its wearer. So at each instant of time, a portable alerting unit worn on a resident or patient has its unique WIFI signature (or WIFI profile), which is consisted of the complete set of e.g. all the signal strengths from all the WIFI routers or at least part of the set at that instant of time.

It is appreciated that determining WIFI profile/signature of a portable alerting unit (e.g. by using its WIFI chip) consumes a lot of battery power of the portable alerting unit, and thus its WIFI profile/signature is not determined or checked continuously, in an embodiment of the present disclosure. As an example, when a resident/patient is lying in a bed (e.g. in her/his bed), or when a resident/patient is not moving very much, her/his portable alerting unit's WIFI profile/signature, and thus its location, will not be determined or checked, in order to preserve its battery power. In consideration of the above, the WIFI profile/signature of a portable alerting unit is determined or checked only when there is a lot of movement or a substantial movement of the portable alerting unit, and such determination or checking is stoped when there is no more (substantial) movement, in an embodiment of the present disclosure. In addition or alternatively, such determination or checking may be started upon a off-bed condition (i.e. getting out of bed) that can be detected by e.g. an orientation unit (such as accelerometer, gyroscope) in a portable alerting unit, in an embodiment of the present disclosure. It is understood that a off-bed condition may be detected by e.g. a specific motion (or orientation or change in orientation) showing a transition from being or lying in bed to getting out of bed. Upon a off-bed condition, the associated portable alerting unit may be configured or instructed to determine or check its WIFI signature/profile, and thus also its location, according to a timing setting (either as predetermined or as instructed) e.g. every 10 minutes for the first few hours, then every half an hour for next few hours, and then every one hour for following few hours, and so on, in an embodiment of the present disclosure.

A portable unit may be lost when being deattached from an absorbent article, due to its small form factor. In order to avoid this, a portable unit is configured to be woken up when being deattached from an absorbent article, then determine its WIFI signature and send it according to a timing setting (either as predetermined or as instructed), e.g. periodically with a period increasing over time. As an example, when a portable unit (e.g. a pod) is taken out of an absorbent article (e.g. a diaper), it is disconnected from the absorbent article, and in turn woken up once in a while based on a predetermined rule or as instructed, and then determines and sends its WIFI signature according to a timing setting (e.g. as predetermined or as instructed), e.g. once every hour, and then less and less, in an embodiment of the present disclosure. In this way, it is possible to find out where a portable unit is, if it is lost.

It is understood that at night, a person generally sleeps in her/his bed and thus does not move very much for a certain period of time. When sleeping at night, some person even spends most of time in one exact location. In consideration of this, it is possible to identify, with aid of the portable alerting unit attached to an absorbent article on a resident or patient, in which room (and/or in which bed if there are more than one bed in that room) that resident or patient sleeps, by comparing the portable alerting unit's WIFI signature (or WIFI profile, e.g . . . its received WIFI signal strength(s)) at night when there is no or less movement being detected to the WIFI signatures (or WIFI profile) of the rooms (and/or of the beds) in the care facility. As an example, a resident or patient is identified as sleeping in a specific room or bed if her/his portable alerting unit's WIFI signature (or WIFI profile, e.g. the WIFI signal strength(s) received by the portable alerting unit worn on that resident or patient) at night with no or less movement being detected (substantially) matches a WIFI signature (or WIFI profile) of that specific room or bed. That is, a room and/or a bed assigned to a resident/patient and/or its particular location in the facility may be identified based on the fact that there is not a lot of movement for a very long period of time during night (i.e. when the resident/patient is sleeping in her/his bed in her/his room, e.g. by WIFI signature (or WIFI profile) of the resident/patient (in particular, of a portable alerting unit worn on her/him) indicating for a long period during night a precise location without a substantial change and without a substantial movement).

According to an embodiment of the present disclosure, in a care facility the WIFI signature (or WIFI profile, e.g. one or more of the WIFI signal strengths from WIFI routers) of a portable alerting unit worn on a resident or patient may be used to locate that resident or patient. In particular, the WIFI signature of a portable alerting unit worn on a resident or patient is compared to the WIFI signatures of the rooms in a care facility, and the resident or patient is determined as being in or close to the room or bed whose WIFI signature matches best the portable alerting unit's WIFI signature, in an embodiment of the present disclosure.

Considering the scenario where the resident of room No. 24 got out of her/his bed, walked out of her/his room No. 24 and got lost in the care facility, as illustrated in FIG. 1. In order to locate her/him, the WIFI signature (or WIFI profile, e.g. one or more of the WIFI signal strengths from WIFI routers) of the portable alerting unit worn on her/him is determined and compared to the WIFI signature of the rooms and/or beds in the care facility. As an non-limiting example, the portable alerting unit carried by the resident of room No. 24 is determined to have a WIFI signature that matches best (e.g. is similar to or same as) the WIFI signature of room No. 5 (e.g. having a strongest signal strength from WIFI B, a slightly weaker strength from WIFI A and C, . . . ), thereby this resident may be determined to be located in or close to room No. 5, as illustrated in FIG. 1.

In an embodiment of the present disclosure, in a facility system, in response to a certain condition e.g. upon detecting an off-bed condition (e.g. motions illustrating a resident/patient gets out of her/his bed), a portable alerting unit worn on the resident/patient sends a message to a server that registers/stores WIFI signature/profile of the facility system (e.g. of the rooms, beds, toilets, and/or residents/patients in the facility), and then the server responds with a message/instruction indicating the timing for the portable alerting unit to provide its WIFI signature/profile, i.e. when and/or how often (e.g. periodically, and in some cases the period may vary (e.g. increase) over time) the portable alerting unit shall determine and send its WIFI signature/profile to the server that in turn implements, based on the WIFI signature/profile received from the portable alerting unit, the process as described in the present disclosure, e.g. determining the location of rooms, beds, toilets, and/or residents/patients in the facility, automatically recording an action on or by an object in the facility, and etc.

In an alternative embodiment of the present disclosure, the setting (i.e. timing such as when and how often) for a portable alerting unit to provide its WIFI signature/profile in response to a certain condition (e.g. an off-bed condition) may be predetermined or preset e.g. during the initiation or set up of the portable alerting unit or the system, rather than being instructed by the server to the portable alerting unit in respond to a certain condition message. In such embodiment of the present disclosure, a portable alerting unit is provided in advance with such setting (i.e. timing) information, and thus the step of the server sending to the portable alerting unit a message/instruction indicating the timing for providing WIFI signature/profile may be omitted, and the portable alerting unit simply determines and sends its WIFI signature/profile to the server according to the predetermined/preset setting (i.e. timing) following the certain condition.

In some embodiment of the present disclosure, a portable alerting unit may also be configured to stop determining and sending its WIFI signature/profile in response to another certain condition, e.g. upon detecting an on-bed condition (e.g. motions illustrating a resident/patient gets on her/his bed).

It is appreciated that a portable alerting unit may be configured to determine and send its WIFI signature/profile to a server in a facility system periodically or in respond to a request form the server, according to an embodiment of the present disclosure. It is also appreciated that the process as described in the present disclosure, e.g. determining the location of rooms, beds, toilets, and/or residents/patients in the facility, automatically recording an action on or by an object in the facility, and etc. may be implemented by either server or portable alerting unit, depending on the particular practice or setting. And based on the outcome of process as described in the present disclosure, server and/or portable alerting unit may alert or prompt to the facility personnel(s) e.g. caregiver(s) accordingly.

It is appreciated that a portable alerting unit may be configured to normally sleep and to wake up periodically to detect and measure the wetness in its associated absorbent article (i.e. the absorbent article on which the portable alerting unit is attached) and provide alert accordingly, in order to preserve battery life. As a non-limiting example, a portable alerting unit is configured to wake up every 20 seconds to check the wetness in its associated absorbent article and alert when necessary, and then go back to sleep.

It is further appreciated that WIFI chip consumes a lot of battery power. Therefore, in a portable alerting unit, WIFI chip wakes up only when necessary, e.g. only when an alert needs to be communicated via WIFI. To this end, even though a portable alerting unit wakes up and check wetness periodically with a short period (e.g. every 20 seconds), it doesnt not necessarily check its WIFI signature/profile every time when it wakes up to check wetness (i.e. with that short period, e.g. every 20 seconds), because WIFI signature/profile can be checked only upon wake-up of a corresponding WIFI chip while a WIFI chip in a portable alerting unit is normally waked up only when necessary, e.g. when an absorbent article with the portable alerting unit being attached is put on or take off, when the portable alerting unit is deattached from an associated absorbent article, and when an alert needs to be send in response to a wetness check. So in an embodiment of the present disclosure, a portable alerting unit may be configured to determine and send its WIFI signature/profile under those particular circumstances.

Also, as mentioned above, a portable alerting unit may be configured to determine and send its WIFI signature/profile e.g. as per a predetermined/preset timing or as instructed by the server, when a certain condition e.g. off-bed condition or a substantial movement is determined, and to stop such determining and sending when a different condition e.g. on-bed condition, no substantial movement for a substantial period of time, and etc is determined.

As an non-limiting example, a portable alerting unit checks wetness e.g. every 20 seconds, but will not check and send its WIFI signature/profile to a server until a certain condition e.g. a off-bed condition or a substantial movement is determined. Upon detecting a certain condition e.g. a off-bed condition or a substantial movement, the portable alerting unit starts to check and send its WIFI signature/profile to a server according to a predetermined/preset timing or as per instructed by the server, e.g. checking and sending its WIFI signature/profile every 10 minutes for the first few (e.g. 2) hours, then every half an hours for the next few (e.g. 2) hours, and then every an hour for the next few (e.g. 2) hours, and so on. In a circumstance where a resident/patient with a portable alerting unit gets out of her/his bed, walks around to another room, and then sit without substantial movement in the other room for 3 hours, her/his portable alerting unit may start, upon detecting her/his off-bed condition, to check and send its WIFI signature/profile according to a predetermined/preset timing or as per instructed by the server, and then stop, when he/she sits without substantial movement in the other room, such checking and sending, as an non-limiting example.

In consideration of the above, the WIFI signature (i.e. WIFI profile) of a portable alerting unit is not continuously monitored in an embodiment of the present disclosure, because such continuous monitoring consumes a lot of battery power and thus the battery will not last long. As an example, according to an embodiment of the present disclosure, a portable alerting unit will not determine its WIFI signature when its wearer is sleeping in the bed and has not move very much. Instead, only when its wearer experiences specific motion(s) e.g. getting out of her/his bed, walking away from the bed, walking out of her/his room, etc., the portable alerting unit starts to check its WIFI signature, e.g. periodically. In order to further reduce the power consumption and preserve battery life, the period for checking WIFI signature may increase over time.

As a non-limiting example, a portable alerting unit does not check its WIFI signature if its orientation determination unit such as gyroscope, accelerometer, etc. determines its wearer (i.e. a resident) is lying in the bed and/or has not move very much. Therefore, e.g. when a resident is sleeping in the bed at night for 8 hours, her/his portable alerting unit does not check its WIFI signature for such a long period of time. However, when specific motion(s) e.g. getting out of the bed, walking away from the bed, walking out of her/his room, etc is recognized by the orientation determination unit, the portable alerting unit starts to check its WIFI signature periodically, e.g. once a minute for the first hour, once every 5 minutes for the next 2 hours, then once every 30 minutes for the next 6 hours, and after that, once a hour. And when another specific motion(s) e.g. getting into the bed or no more substantial movement is recognized by the orientation determination unit, the portable alerting unit stops checking its WIFI signature. Thereby, it is possible to track a resident's location when she/he gets out of her/his bed (off-bed condition) and/or walks out of her/his room (away condition), while reducing power consumption and preserving battery life.

It is understood that a care facility e.g. nursing center is often subsidized by the government or some program or organization, e.g. receives revenue from the government or some program or organization, but under certain condition. As an example, a resident who needs help or assistance with toileting resides in a care facility, in which case when this resident wants toileting, the facility (in particular, one of its employees) needs take the resident physically, place her/him on the toilet, clean her/him after toileting, and bring her/him back e.g. to her/his bed. And the subsidies from government or program or organization are for all these assistances or actions, and therefore the care facility is required to record their assistances or actions in order to receive the subsidies. That is, a care facility, in particular its employees, needs to keep record after each assistance or actions, in order to receive subsidies. However, sometimes they forget to keep record, which might result in some problem or difficulty with subsidies.

According to an embodiment of the present disclosure, a location based recording method and system are provided, which automatically records or registers certain action(s) e.g. assistance for toileting. As an example, a location based recording method and system record or register when and/or how many times certain action(s) is conducted and/or the subject of the certain action(s) (i.e. on whom the action(s) is conducted) and/or the subject conducting the certain action(s) (i.e. who conducted the action(s)).

In a care facility, each room generally is equipped with a toilet such that a resident can do toileting and take shower without leaving her/his room. In FIG. 1, a toilet 3′ is schematically illustrated, which is provided in a room No. 3. It is understood that a toilet inside a room has a different WIFI signature (or WIFI profile, e.g. signal strengths from WIFI routers) than the rest of the room (e.g. than the bed(s) in the room), which can be used to identify and thus automatically record or register the toileting and/or the assistance for toileting. For each toilet in a care facility, its WIFI signature is determined and/or recorded, e.g. by bring a portable alerting unit in that toilet and determining its WIFI signature in that toilet. According to an embodiment of the present disclosure, a location based recording method and system is initiated in response to specific motion(s) of a resident (e.g. detected by motion sensor(s)) or her/his call for assistance. As an example, a resident's desire of toileting may be determined and recorded in response to her/his specific motion(s) (e.g. in response to specific motion signal(s) from motion sensor(s)) or to her/his call for assistance of toileting. Then the WIFI signature (or signal profile) of the portable alerting unit worn on her/him is monitored, and a toileting or an assistance of toileting is recorded or registered if the portable alerting unit worn on the resident experiences a WIFI signature (or WIFI profile) variation that matches the wandering (i.e. trajectory) from the resident's bed to her/his toilet and then back to her/his bed. When the resident is determined as being back in her/his bed, the portable alerting unit stops monitoring its WIFI signature.

As an non-limiting example, a resident/patient with her/his portable alerting unit experiences an out-of-bed motion, e.g. she/he gets out of her/his bed by herself/himself or with aid of someone else (e.g. a caregiver), which may be detected by her/his portable alerting unit who in turn informs a server or a recording/registering means (either in hardware or in software, e.g. in some cases, in the form of a reporting software) of the resident/patient's off-bed condition and starts to check and send its WIFI signature according to a timing setting (either as predetermined or as instructed by the server), and then the sever or the recording/registering means determines based on the WIFI signatures received from that portable alerting unit that the resident/patient is doing toileting (i.e. moving to her/his toilet, doing toileting in her/his toilet, and moving back to her/his bed) and thus records or registers this toileting e.g. in a specific register, e.g. by entering it into a report.

It is appreciated that the same may apply on other actions, e.g. feeding or entertaining. As an example, a caregiver may walk or take a resident/patient to a dining room or an entertainment room, feed the resident/patient or show the resident/patient a TV program there for a period of time, and then walk or take the resident/patient back to her/his room/bed, in which circumstance a similar process as that as described above with respect to toileting may be conducted, in order to automatically register or record the feeding or entertaining in the facility.

It is also appreciated that some other actions e.g. change of absorbent article (such as diaper), or cleaning a resident may be automatically recorded or registered, according to an embodiment of the present disclosure. As an example, a caregiver may remove an absorbent article e.g. a soiled diaper from a resident and put on a clean and dry absorbent article, which can be detected by means of a portable unit (e.g. pod) configured to be attached (e.g. releasably) to absorbent article and will be automatically register or record e.g. in a server or a recording/registering means (such as a reporting software). As another example, a portable unit (e.g. pod) carried by a resident (e.g. attached to her/his absorbent article) may be configured to detect specific movement(s) associated with an action e.g. cleaning her/his body, and initiate an automatic recording or registration of the action e.g. in a server or a recording/registering means (such as a reporting software).

The invention claimed is:

1. A method for locating a portable unit based on WIFI signature, comprising:
the portable unit determining and sending its WIFI signature to a server, and the portable unit's location being determined based on the WIFI signature,
wherein when detecting a motion, the portable unit determines and sends its WIFI signature more frequently than when detecting no motion.

2. The method according to claim 1, further comprising automatically recording an action on or by an object based on WIFI signature, wherein the object carries the portable unit, wherein the action is identified as a sequence of motions, and wherein the automatically recording comprises:
monitoring the WIFI signature of the portable unit;
determining the motion path of the object based on the WIFI signature as monitored;
comparing the determined motion path of the object to a predetermined motion path of the action; and
recording the action if the determined motion path substantially matches the predetermined motion path.

3. The method according to claim 2, wherein the purpose of the recording is to report the recorded action in order for funding.

4. The method according to claim 1, further comprising automatically recording an action on or by an object, wherein the portable unit is carried on the object, wherein the action is identified as a sequence of motions, and wherein the automatically recording comprises:
detecting the action with aid of the portable unit; and
recording the action,
wherein the action comprises change of absorbent article, or cleaning the object, or
the object cleaning another object.

5. The method according to claim 1, wherein the portable unit is attachable to an absorbent article, and comprises:
a WIFI circuit configured to determine and send WIFI signature,
wherein the WIFI circuit is woken up from Sleep mode when the portable unit is detached from the absorbent article, then determines and sends WIFI signature periodically with a period increasing over time, and wherein the WIFI circuit returns to Sleep mode after a certain period of time following its wake up.

6. A method for locating a portable unit based on WIFI signature, comprising:
the portable unit starting to determine and send its WIFI signature to a server when detecting an off-bed condition, and
the portable unit's location being determined based on the WIFI signature.

7. A portable unit attachable to an absorbent article, comprising:
a wetness detection circuit configured to detect wetness in the absorbent article periodically while the portable unit is attached to the absorbent article, and
a WIFI circuit configured to determine and send WIFI signature,
wherein the WIFI circuit is woken up from Sleep mode under a first condition, and
returns to Sleep mode under a second condition; and
wherein the first condition comprises one of:
the absorbent article with the portable article being attached thereto is being put on or taken off,
the portable unit is being detached from the absorbent article,
an alert needs to be sent via WIFI based on the wetness detected by the wetness detection circuit, or
a motion is determined, and
wherein the second condition comprises one of:
a different motion is determined, or
no motion is determined at least for a certain period of time.

8. A method for locating an object inside a building based on WIFI signature, wherein the building is equipped with a number of WIFI routers, and wherein the WIFI signature is one or more of WIFI characteristics with respect to one or more of the WIFI routers, the method comprising:
determining the WIFI signature of a portable unit carried on the object;
comparing the WIFI signature of the portable unit to each of the WIFI signatures of specific locations in the building; and
determining the object to be located at or close to the one specific location whose WIFI signature matches best the WIFI signature of the portable unit.

9. The method according to claim 8, wherein the WIFI signature of the portable unit is determined periodically after specific motion(s) of the portable unit or of the object is detected.

10. The method according to claim 9, wherein the period for determining the WIFI signature of the portable unit increases over time.

11. The method according to claim 8, wherein the building comprises a care facility, and wherein the object comprises a resident in the care facility.

12. The method according to claim 8, further comprising automatically recording an action on or by the object, wherein the action is identified as a sequence of motions, and wherein the automatically recording comprises:
initiating a monitoring on the WIFI signature of the portable unit in response to specific motion(s) of the object or in response to a signal from the object;
determining the motion path of the object based on the WIFI signature as monitored;
comparing the determined motion path of the object to a predetermined motion path; and
recording the action if the determined motion path substantially matches the predetermined motion path.

13. The method according to claim 12, wherein the monitoring on the WIFI signature of the portable unit is terminated after the action is recorded and/or when the object is back to its previous location when the monitoring was initiated.

14. The method according to claim 12, wherein the action on the object comprises assistance for the object with toileting, or walking/moving the object.

15. A system for locating an object inside a building based on WIFI signature, comprising:
a number of WIFI routers deployed in the building, wherein the WIFI signature is one or more of WIFI characteristics with respect to one or more of the WIFI routers;
a portable unit carried on the object; and
a locating device configured to obtain the WIFI signature of the portable unit, compare the WIFI signature of the portable unit to each of the WIFI signatures of specific locations in the building, and determine the object to be located at or close to the one specific location whose WIFI signature matches best the WIFI signature of the portable unit.

16. The system according to claim 15, wherein the locating device obtains the WIFI signature of the portable unit periodically after specific motion(s) of the portable unit or of the object is detected.

17. The system according to claim 16, wherein the period for obtaining the WIFI signature of the portable unit increases over time.

18. The system according to claim 15, wherein the building comprises a care facility, and wherein the object comprises a resident in the care facility.

19. The system according to claim 15, further comprising:
a recording device configured to obtain the WIFI signature of the portable unit as monitored in response to specific motion(s) of or a signal from the object, determine the motion path of the object based on the WIFI signature as monitored, compare the determined motion path of the object to a predetermined motion path, and record an action if the determined motion path substantially matches the predetermined motion path, wherein the action is identified as a sequence of motions.

20. The system according to claim 19, wherein the monitoring on the WIFI signature of the portable unit is terminated after the action is recorded and/or when the object is back to its previous location when the monitoring was initiated.

21. The system according to claim 19, wherein the action on the object comprises assistance for the object with toileting, or walking/moving the object.
